SEQUENCE LISTING (13 sequences)

SEQ ID NO: 1 - protein, 10 aa, synthetic construct
GFTFSDYGMG

SEQ ID NO: 2 - protein, 17 aa, synthetic construct
SISWDSSSKEYADSVKG

SEQ ID NO: 3 - protein, 14 aa, synthetic construct
AWIAIIGGGAHFDY

SEQ ID NO: 4 - protein, 11 aa, synthetic construct
RASQSVSSHLA

SEQ ID NO: 5 - protein, 7 aa, synthetic construct
LTSSLQS

SEQ ID NO: 6 - protein, 9 aa, synthetic construct
MQSKSLPFT

SEQ ID NO: 7 - protein, 123 aa, synthetic construct
EVQLVESGGGLVQPGGSLRLSCAASGFTFSDYGMGWVRQAPGKGLEWVSSISWDSSSKEYADSVKGRFTISRDNSKNTLYLQMNSLRAEDTAVYYCARAWIAIIGGGAHFDYWGQGTLVTVSS

SEQ ID NO: 8 - protein, 108 aa, synthetic construct
DIQMTQSPSSLSASVGDRVTITCRASQSVSSHLAWYQQKPGKAPKLLIYLTSSLQSGVPSRFSGSGSGTDFTLTISSLQPEDFATYYCMQSKSLPFTFGQGTKVEIKR

SEQ ID NO: 9 - protein, 534 aa, synthetic construct
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 10 - protein, 534 aa, synthetic construct
MLHRRGSPGMGVHVGAALGALWFCLTGALEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSITITPQRSPTGAVEVQVPEDPVVALVGTDATLRCSFSPEPGFSLAQLNLIWQLTDTKQLVHSFTEGRDQGSAYANRTALFLDLLAQGNASLRLQRVRVADEGSFTCFVSIRDFGSAAVSLQVAAPYSKPSMTLEPNKDLRPGDTVTITCSSYRGYPEAEVFWQDGQGAPLTGNVTTSQMANEQGLFDVHSVLRVVLGANGTYSCLVRNPVLQQDAHGSVTITGQPMTFPPEALWVTVGLSVCLVALLVALAFVCWRKIKQSCEEENAGAEDQDGEGEGSKTALQPLKHSDSKEDDGQELA

SEQ ID NO: 11 - protein, 316 aa, synthetic construct
MLRGWGGPSVGVSMGTALGVLCLCLTGAVEVQVSEDPVVALVDTDATLRCSFSPEPGFSLRQLNLIWQLTDTKQLVHSFTEGRDQGSAYANRTALFPDLLVQGNASLRLQRVRVTDEGSYTCFVSIQDFDSAAVSLQVAAPYSKPSMTLEPNKDLRPGDMVTITCSSYQGYPEAEVFWKDGQGLPLTGNVTTSQMANERGLFDVHSVLRVVLGANGTYSCLVRNPVLQQDAHGSVTITGQPMTFPPEALWVTVGLSVCLVILLVALAFVCWRKIKQSCEEENAGAEDQDGDGEGSKTALRPLKHSENKEDDGQEIA

SEQ ID NO: 12 - protein, 316 aa, synthetic construct
MLRGWGGPSVGVCVRTALGVLCLCLTGAVEVQVSEDPVVALVDTDATLRCSFSPEPGFSLAQLNLIWQLTDTKQLVHSFTEGRDQGSAYSNRTALFPDLLVQGNASLRLQRVRVTDEGSYTCFVSIQDFDSAAVSLQVAAPYSKPSMTLEPNKDLRPGNMVTITCSSYQGYPEAEVFWKDGQGVPLTGNVTTSQMANERGLFDVHSVLRVVLGANGTYSCLVRNPVLQQDAHGSVTITGQPLTFPPEALWVTVGLSVCLVVLLVALAFVCWRKIKQSCEEENAGAEDQDGDGEGSKTALRPLKPSENKEDDGQEIA

SEQ ID NO: 13 - protein, 205 aa, synthetic construct
EDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITG

(19) United States
(12) Patent Application Publication
(10) Pub. No.: US 2025/0197501 A1
(43) Pub. Date: Jun. 19, 2025

(54) RECOMBINANT ANTIBODY, IMMUNOCONJUGATE COMPRISING THE SAME, AND USES THEREOF IN TREATING CANCERS

(71) Applicant: Development Center for Biotechnology, New Taipei City (TW)

(72) Inventors: Chu-Bin LIAO, Taipei (TW); Chen-Jei HONG, Taipei (TW); Shih-Chong TSAI, Taipei (TW); Chien-Yu LIN, Taipei (TW); Yu-Ping CHOU, Taipei (TW); Cheng-Chou YU, Taipei (TW); Wei-Ting SUN, Taipei (TW)

(21) Appl. No.: 18/544,542

(22) Filed: Dec. 19, 2023

(51) Int. Cl. C07K 16/28; A61K 39/00; A61K 47/68; A61K 51/10; A61P 35/00

(52) U.S. Cl. CPC C07K 16/2827; A61K 47/68031; A61K 47/68033; A61K 47/68035; A61K 47/6813; A61K 47/6815; A61K 47/6849; A61K 51/1027; A61P 35/00; A61K 2039/505; C07K 2317/24

(57) ABSTRACT

Disclosed herein is a recombinant antibody or a fragment thereof exhibiting binding affinity and specificity toward B7-H3. According to some embodiments of the present disclosure, the recombinant antibody or its fragment comprises a heavy chain variable (VH) domain and a light chain variable (VL) domain respectively having the amino acid sequences of SEQ ID NOs: 7 and 8. Also disclosed herein are an immunoconjugate comprising the recombinant antibody or its fragment, and a method of treating cancers by use of the present immunoconjugate.

### SEQUENCE LISTING XML

The present application is being filed along with a Sequence Listing XML in electronic format. The Sequence Listing XML is provided as an XML file entitled P4309_SEQ_AF, created Nov. 28, 2023, which is 14 Kb in size. The information in the electronic format of the Sequence Listing XML is incorporated herein by reference in its entirety.

### BACKGROUND OF THE INVENTION

### 1. Field of the Invention

The present disclosure in general relates to the field of disease treatment. More particularly, the present disclosure relates to a novel antibody and uses thereof in the treatment of cancers.

### 2. Description of Related Art

Cancer is the major cause of death. According to International Agency for Research on Cancer (IARC), there are estimated 10 million deaths and 19.3 million new cases in 2020 globally. Despite the global effort to develop novel cancer therapies and improve current cancer treatment efficacy, the mortality rate of cancer remains high. While different types of therapeutic approaches are currently available, including chemotherapy, surgery, radiation therapy, hormone therapy, biologic therapy, targeted therapy, and most recently, immunotherapy and cell-based therapy, cancer still poses significant health risks and increases the economic burden.

The growth of tumor depends on the complex tissue environment providing a milieu that sustains tumor cell growth and provides blood supply through angiogenesis. On the other hand, tumor growth and metastasis also depend on their capacity to evade host immune surveillance and overcome host defenses. It has been extensively studied that most tumors express antigens (also known as “tumor-associated antigens” (TAAs)) that can be recognized to a variable extent by the host immune system due to weakly immunogenicity. However, in most cases, the effectiveness of the tumor immunity is insufficient to suppress tumor growth due to the development of escape mechanisms that allow cancer cells to block the immune system, such as upregulating the expression of programmed cell death ligand 1 (PD-L1) to modulate the immune system, releasing immunosuppressive factors and recruiting immunosuppressive cells to the tumor environment.

B7-H3, also known as CD276, is a type I transmembrane protein composed of an extracellular domain, a single transmembrane domain, and a short intracellular domain. B7-H3 belongs to the B7 family members, which are immunoglobulin (Ig) superfamily with an immunoglobulin-V-like and an immunoglobulin-C-like domain (e.g., IgV-IgC). The majority human form of B7-H3 contains two extracellular tandem IgV-IgC domains (4Ig-B7-H3). In mice and rat, only 2 Ig domains (IgV-IgC; 2Ig-B7-H3) was reported and exhibits similar function as human 4Ig-B7-H3.

B7-H3 is known to be overexpressed in various types of cancers, including non-small-cell lung cancer, hypopharyngeal carcinoma, oral cancer, kidney cancer, urothelial carcinoma, colorectal cancer, prostate cancer, glioblastoma multiforme, ovarian cancer, and pancreatic cancer. In particular, it is reported that in prostate cancer, the expression of B7-H3 positively correlates with malignancy and cancer progression. Similarly, in head and neck cancers, B7-H3 is detected as high expression and correlates with poor survival; and in pancreatic cancer and ovarian cancer, the expression of B7-H3 correlates with lymph node metastasis and pathological progression. Besides, in B7-H3-positive cancer cell lines, the treatment of small interfering RNA (siRNA) against B7-H3 could reduce cancer cell migration and invasion. Therefore, B7-H3 may serve as a potential drug target for development of therapeutic antibody or antibody drug conjugate (ADC) in the treatment of cancers.

Several anti-B7-H3 antibodies are developed in the art; however, their safety and efficacy in patients are still being determined by ongoing clinical trials. Further, most of the anti-B7-H3 antibodies are murine antibodies or humanized antibodies that usually exhibit high immunogenicity leading to adverse effect in human subjects. In view of the foregoing, there is a continuing interest in developing a novel antibody exhibiting binding specificity toward B7-H3 so as to treat cancers in a more efficient and safer manner.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, the first aspect of the disclosure is directed to a B7-H3 targeting recombinant antibody (i.e., a recombinant antibody exhibiting binding affinity and specificity towards B7-H3, and capable of targeting B7-H3), or a fragment thereof (e.g., single-chain variable fragment, scFv). In structure, the B7-H3 targeting recombinant antibody or antibody fragment comprises a heavy chain variable (VH) domain and a light chain variable (VL) domain, in which the VH domain comprises a first heavy chain complementarity determining region (CDR-H1), a second heavy chain CDR (CDR-H2) and a third heavy chain CDR (CDR-H3), and the VL domain comprises a first light chain CDR (CDR-L1), a second light chain CDR (CDR-L2) and a third light chain CDR (CDR-L3).

According to certain embodiments of the present disclosure, the CDR-H1, CDR-H2 and CDR-H3 respectively comprise the amino acid sequences of SEQ ID NOs: 1, 2 and 3, and the CDR-L1, CDR-L2 and CDR-L3 respectively comprise the amino acid sequences of SEQ ID NOs: 4, 5 and 6.

According to some embodiments of the present disclosure, the VH and VL domains of the B7-H3 targeting recombinant antibody or antibody fragment respectively comprise the amino acid sequences at least 85% identical to SEQ ID NOs: 7 and 8; preferably, at least 90% identical to SEQ ID NOs: 7 and 8; more preferably, at least 95% identical to SEQ ID NOs: 7 and 8. In one exemplary example of the present disclosure, the VH and VL domains of the B7-H3 targeting recombinant antibody or its fragment respectively comprise the amino acid sequences of SEQ ID NOs: 7 and 8 (i.e., the amino acid sequences 100% identical to SEQ ID NOs: 7 and 8).

The second aspect of the present disclosure pertains to the uses of the B7-H3 targeting recombinant antibody or its fragment of the present disclosure in the preparation of an immunoconjugate for treating cancers in a subject. According to the embodiments of the present disclosure, the immunoconjugate comprises the present B7-H3 targeting recombinant antibody or antibody fragment, a therapeutic agent, and a linker connecting the therapeutic agent to the B7-H3 targeting recombinant antibody or antibody fragment. Depending on desired purpose, the therapeutic agent may be a cytotoxic drug, a radioactive nuclide, a cytokine, a hormone drug, an immune stimulating agent, or an immunotherapeutic drug.

According to some preferred embodiments of the present disclosure, the therapeutic agent is a cytotoxic drug, for example, auristatin or its derivative. In one exemplary embodiment, the therapeutic agent is monomethyl auristatin E (MMAE). In another embodiment, the therapeutic agent is monomethyl auristatin F (MMAF).

Also disclosed herein is a pharmaceutical composition for treating cancers. The pharmaceutical composition comprises the immunoconjugate of the present disclosure; and optionally, a pharmaceutically acceptable carrier.

Another aspect of the present disclosure pertain to a method of treating a cancer in a subject. The method comprises administering to the subject an effective amount of the immunoconjugate or pharmaceutical composition of the present disclosure.

Examples of the cancer treatable with the present method include, but are not limited to, breast cancer, gastric cancer, colorectal cancer, gallbladder cancer, prostate cancer, cervical cancer, ovarian carcinoma, chronic or acute lymphocytic leukemia, bladder cancer, renal cancer, hepatocellular carcinoma, head and neck squamous cell carcinoma, glioblastoma, esophageal cancer, pancreatic cancer, oral cancer, lung cancer, melanoma and lymphoma.

The subject is a mammal; preferably, a human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings briefly discussed below.

FIG. 1 is the result of enzyme-linked immunosorbent assay (ELISA) that depicts the binding activity of antibody 91H06 to specified proteins according to Example 1.1 of the present disclosure, in which the antibody 91H06 exhibits binding specificity towards B7-H3, without cross-reacting with other B7 family proteins.

FIG. 2 are the results of flow cytometry that depict the binding affinities of antibody Ifinatamab and 91H06 to B7-H3 positive cells according to Example 1.2 of the present disclosure. Panel (A): Binding affinity of antibody Ifinatamab to EMT6 cells. Panel (B): Binding affinity of antibody 91H06 to EMT6 cells. Panel (C): Binding affinity of antibody Ifinatamab to ID8 cells. Panel (D): Binding affinity of antibody 91H06 to ID8 cells. The cell binding activities of tested antibodies were analyzed by flow cytometry (filled peaks). Isotype control antibodies (open peaks) were used to demonstrate the specificity of staining.

FIG. 3 is a line chart depicting the tumor volume of Detroit 562 tumor-bearing mice administered with specified treatments according to Example 3 of the present disclosure. Vehicle: The tumor-bearing mice administered with 2-(N-morpholino) ethanesulfonic acid (MES) buffer one every week for three consecutive weeks. 1.5 mpk: The tumor-bearing mice administered with 1.5 mg/kg of 91H06-MMAE once every week for three consecutive weeks, 5 mpk: The tumor-bearing mice administered with 5 mg/kg of 91H06-MMAE once every week for three consecutive weeks.

FIG. 4 is a line chart depicting the tumor volume of FaDu tumor-bearing mice administered with specified treatments according to Example 3 of the present disclosure. Vehicle: The tumor-bearing mice administered with single dose of MES buffer. 1.5 mpk: The tumor-bearing mice administered with single dose of 91H06-MMAE at 1.5 mg/kg. 5 mpk: The tumor-bearing mice administered with single dose of 91H06-MMAE at 5 mg/kg.

FIG. 5 is a line chart depicting the tumor volume of Detroit 562 tumor-bearing mice administered with 91H06-MMAE at 5 mg/kg (mpk) once every week for three consecutive weeks according to Example 3 of the present disclosure.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “antibody” (Ab) is used in the broadest sense and specifically covers monoclonal antibodies (including full length monoclonal antibodies), polyclonal antibodies, multi-specific or multivalent antibodies (e.g., bi-specific antibodies), chimeric antibodies, humanized antibodies and antibody fragments so long as they exhibit the desired biological activity. The term “antibody fragment” or “the fragment of an antibody” refers to a portion of a full-length antibody, generally the antigen binding or variable domain (i.e., VH and VL domains) of a full-length antibody. Examples of the antibody fragment include fragment antigen-binding (Fab), Fab′, F(ab′)₂, single-chain variable fragment (scFv), diabody, linear antibody, single-chain antibody molecule, and multi-specific antibody formed from antibody fragments. Depending on the antibody amino acid sequence of the constant domain of its heavy chains, immunoglobulins can be assigned to different classes, including immunoglobulin G (IgG), immunoglobulin A (IgA), immunoglobulin M (IgM), immunoglobulin D (IgD) or immunoglobulin E (IgE), in which some classes may be further divided into subclasses (isotypes), such as IgG1, IgG2, IgG3, IgG4, IgA1 and IgA2. The heavy-chain constant domains that correspond to the different classes of immunoglobulins are called alpha (α), gamma (γ), delta (δ), epsilon (ε), and mu (μ), respectively. The subunit structures and three-dimensional configurations of different classes of immunoglobulins are well known in the art, for example, Abbas et al., “Cellular and Molecular Immunology,” 4th ed. (2000). An antibody may be part of a larger fusion molecule, formed by covalent or non-covalent association of the antibody with one or more other proteins or peptides.

As used herein, the term “monoclonal antibody” (mAb) refers to an antibody obtained from a population of substantially homogeneous antibodies. In contrast to polyclonal antibodies which may include different antibodies directed to different epitopes, each monoclonal antibody is directed against a single determinant (i.e., epitope) on the antigen. Monoclonal antibodies may be produced by fusing a normally short-lived, antibody-producing B cell to a fast-growing cell, such as an immortal cell. The resulting hybrid cell, or hybridoma, multiplies rapidly, creating a clone that produces large quantities of the antibody. Alternatively, monoclonal antibodies may also be produced by recombinant DNA methods, in which a portion of the heavy and/or light chain is identical with or homologous to corresponding sequences in antibodies derived from a species or belonging to an antibody class or subclass, while the remainder of the chain identical with or homologous to corresponding sequences in antibodies derived from another species or belonging to another antibody class or subclass, as well as fragments of such antibodies, as long as they exhibit the desired biological activity.

As used herein, the term “recombinant antibody” refers to an antibody that is expressed and isolated from a cell or cell line transfected with an expression vector (or possibly more than one expression vector, typically two expression vectors) comprising the coding sequence of the antibody, where said coding sequence is not naturally associated with the cell.

The term “complementarity determining region” (CDR) used herein refers to the hypervariable region of an antibody molecule that forms a surface complementary to the three-dimensional surface of a bound antigen. Proceeding from N-terminus to C-terminus, each of the antibody heavy and light chains comprises three CDRs (CDR-1, CDR-2 and CDR-3). An antigen combining site, therefore, includes a total of six CDRs that comprise three CDRs in the variable domain of a heavy chain (i.e., CDR-H1, CDR-H2 and CDR-H3), and three CDRs in the variable domain of a light chain (i.e., CDR-L1, CDR-L2 and CDR-L3).

The “variable domain” of an antibody refers to the amino-terminal domains of heavy or light chain of the antibody. These domains are generally the most variable parts of an antibody and contain the antigen-binding sites. The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)).

As discussed herein, minor variations in the amino acid sequences of antibody (especially minor variations in the FR sequences of antibody) are contemplated as being encompassed by the presently disclosed and claimed inventive concept(s), providing that the variations in the amino acid sequence maintain at least 85% sequence identity, such as at least 85%, 86%, 87%, 88%, 89%, 90%, 91%, 92%, 93%, 94%, 95%, 96%, 97%, 98% and 99% sequence identity. The antibody of the present disclosure may be modified specifically to alter a feature of the antibody unrelated to its physiological activity. For example, certain amino acid residues in the framework (FR) region of the antibody can be changed and/or deleted without affecting the physiological activity of the antibody in this study (i.e., its ability to treat cancers). In particular, conservative amino acid replacements are contemplated. Conservative replacements are those that take place within a family of amino acid residues that are related in their side chains. Genetically encoded amino acid residues are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid residue within the antigen-biding sites, i.e., CDRs. Whether an amino acid change results in a functional peptide can readily be determined by assaying the specific activity of the peptide derivative. Fragments or analogs of proteins/peptides can be readily prepared by those of ordinary skill in the art. Preferred amino- and carboxy-termini of fragments or analogs occur near boundaries of functional domains.

“Percentage (%) sequence identity” is defined as the percentage of amino acid residues in a candidate sequence that are identical with the amino acid residues in the specific peptide sequence, after aligning the sequences and introducing gaps, if necessary, to achieve the maximum percent sequence identity, and not considering any conservative substitutions as part of the sequence identity. Alignment for purposes of determining percentage sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, sequence comparison between two amino acid sequences was carried out by computer program Blastp (protein-protein BLAST) provided online by Nation Center for Biotechnology Information (NCBI). The percentage amino acid sequence identity of a given amino acid sequence A to a given amino acid sequence B (which can alternatively be phrased as a given amino acid sequence A that has a certain % amino acid sequence identity to a given amino acid sequence B) is calculated by the formula as follows:

$$\frac{X}{Y} \times 100$$

where X is the number of amino acid residues scored as identical matches by the sequence alignment program BLAST in that program's alignment of A and B, and where Y is the total number of amino acid residues in A or B, whichever is shorter.

As used herein, the terms “link”, “couple”, “conjugate” and “connect” are used interchangeably to refer to any means of connecting two components either via direct linkage or via indirect linkage between two components.

As used herein, the term “treat,” “treating” and “treatment” are interchangeable, and encompasses partially or completely preventing, ameliorating, mitigating and/or managing a symptom, a secondary disorder or a condition associated with cancers. The term “treating” as used herein refers to application or administration of the antibody or immunoconjugate of the present disclosure to a subject, who has a symptom, a secondary disorder or a condition associated with cancers, with the purpose to partially or completely alleviate, ameliorate, relieve, delay onset of, inhibit progression of, reduce severity of, and/or reduce incidence of one or more symptoms, secondary disorders or features associated with cancers. Symptoms, secondary disorders, and/or conditions associated with cancers include, but are not limited to, nausea, vomiting, loss of appetite, bowel changes, constipation, fatigue, muscle weakness, broken bones, swelling or lump, blooding, cough, fever, neurological problems (e.g., seizures, vision changes, hearing change or drooping of the face), body weight changes (i.e., weight gain or weight loss), coma and pain. Treatment may be administered to a subject who exhibits only early signs of such symptoms, disorder, and/or condition for the purpose of decreasing the risk of developing the symptoms, secondary disorders, and/or conditions associated with cancers. Treatment is generally “effective” if one or more symptoms or clinical markers are reduced as that term is defined herein. Alternatively, a treatment is “effective” if the progression of a symptom, disorder or condition is reduced or halted.

The term “effective amount” as referred to herein designate the quantity of a component which is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two, or more doses in a suitable form to be administered at one, two or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amount may be expressed, for example, in grams, milligrams or micrograms or as milligrams per kilogram of body weight (mg/Kg). Alternatively, the effective amount can be expressed in the concentration of the active component (e.g., the immunoconjugate of the present disclosure), such as molar concentration, mass concentration, volume concentration, molality, mole fraction, mass fraction and mixing ratio. Persons having ordinary skills could calculate the human equivalent dose (HED) for the medicament (such as the present immunoconjugate) based on the doses determined from animal models. For example, one may follow the guidance for industry published by US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

The terms “subject” refers to an animal including the human species that is treatable by the recombinant antibody, antibody fragment, immunoconjugate, pharmaceutical composition and/or method of the present invention. The term “subject” is intended to refer to both the male and female gender unless one gender is specifically indicated.

### II. DESCRIPTION OF THE INVENTION

### (i) Recombinant Antibody

The first aspect of present disclosure provides an anti-B7-H3 monoclonal antibody (mAb) designated as mAb “91H06”. According to the embodiments of the present disclosure, the present mAb is characterized by exhibiting a binding affinity and specificity to human B7-H3 protein, and is capable of efficiently trigger cellular internalization of the surface B7-H3 protein.

According to some embodiments of the present disclosure, the present mAb 91H06 is produced by phage-displayed scFv libraries. As would be appreciated, the present mAb 91H06 may alternatively be produced by conventional immunization method (i.e., immunizing animals with a specific peptide to induce the animal producing peptide-specific Abs), or recombinant DNA technology (also known as DNA cloning technology; i.e., constructing and transducing a recombinant DNA encoding a specific Ab into a host cell thereby expressing the Ab).

In structure, the mAb 91H06 comprises three CDRs in the VH domain thereof (i.e., CDR-H1, CDR-H2, and CDR-H3), and three CDRs in the VL domain thereof (i.e., CDR-L1, CDR-L2, and CDR-L3).

According to some embodiments of the present disclosure, the CDR-H1, CDR-H2, and CDR-H3 of mAb 91H06 respectively comprise the amino acid sequences of “GFTFSDYGMG” (SEQ ID NO: 1), “SISWDSSSKEYADSVKG” (SEQ ID NO: 2) and “AWIAIIGGGAHFDY” (SEQ ID NO: 3); and the CDR-L1, CDR-L2, and CDR-L3 of mAb 91H06 respectively comprise the amino acid sequences of “RASQSVSSHLA” (SEQ ID NO: 4), “LTSSLQS” (SEQ ID NO: 5) and “MQSKSLPFT” (SEQ ID NO: 6).

As an example, the amino acid sequences of the VH and VL domains of mAb 91H06 are respectively provided as SEQ ID NOs: 7 and 8, described below, in which the CDRs (i.e., the CDR-H1, CDR-H2 and CDR-H3 of VH domain, and the CDR-L1, CDR-L2 and CDR-L3 of VL domain) are marked in bold letters, in sequence.

(VH domain of mAb 91H06)
SEQ ID NO: 7
EVOLVESGGGLVQPGGSLRLSCAASGFTFSDYGMGWVRQAPGKGLEWVSS
ISWDSSSKEYADSVKGRFTISRDNSKNTLYLQMNSLRAEDTAVYYCARAW
IAIIGGGAHFDYWGQGTLVTVSS
(VL domain of mAb 91H06)
SEQ ID NO: 8
DIQMTQSPSSLSASVGDRVTITCRASQSVSSHLAWYQQKPGKAPKLLIYL
TSSLQSGVPSRFSGSGSGTDFTLTISSLQPEDFATYYCMQSKSLPFTFGQ
GTKVEIKR

Since the binding affinity and specificity of an antibody are mainly determined by the CDR sequences thereof, as could be appreciated, the framework (FR) sequences of the VH and VL domains may vary (e.g., being substituted by conserved or non-conserved amino acid residues) without affecting the binding affinity and/or specificity of the present antibody. Preferably, the FR sequence is conservatively substituted by one or more suitable amino acid(s) with similar properties; for example, the substitution of leucine (an nonpolar amino acid residue) by isoleucine, alanine, valine, proline, phenylalanine, or tryptophan (another nonpolar amino acid residue); the substitution of aspartate (an acidic amino acid residue) by glutamate (another acidic amino acid residue); or the substitution of lysine (an basic amino acid residue) by arginine or histidine (another basic amino acid residue).

Based on the conservative substitution, a skilled artisan may substitute the amino acid residue(s) of the FR sequences of the VH and VL domains of mAb 91H06 without affecting its activity and/or effect (i.e., binding to B7-H3 and/or triggering the internalization of B7-H3). Accordingly, the antibody comprising substituted amino acid(s) in its FR sequences of VH and VL domains are intended to be included within the scope of the present disclosure. According to certain embodiments, the VH domain of mAb 91H06 comprises an amino acid sequence at least 85% (i.e., 85%, 86%, 87%, 88%, 89%, 90%, 91%, 92%, 93%, 94%, 95%, 96%, 97%, 98%, 99% or 100%) identical to SEQ ID NO: 7, and the VL domain of mAb 91H06 comprises an amino acid sequence at least 85% (i.e., 85%, 86%, 87%, 88%, 89%, 90%, 91%, 92%, 93%, 94%, 95%, 96%, 97%, 98%, 99% or 100%) identical to SEQ ID NO: 8. According to some preferred embodiments, the VH and VL domains of mAb 91H06 respectively comprise the amino acid sequences at least 90% identical to SEQ ID NOs: 7 and 8. More preferably, the VH and VL domains of mAb 91H06 respectively comprise the amino acid sequences at least 95% identical to SEQ ID NOs: 7 and 8.

Depending on intended purpose, the present mAb 91H06 may be produced in the form of IgG, IgA, IgM, IgD or IgE. According to some exemplary embodiments, the present mAb is produced in the form of IgG, which comprises a pair of Fc regions, and a pair of Fab regions respectively linked to the N-termini of the pair of Fc regions.

According to certain embodiments of the present disclosure, the mAb 91H06 is a fully human anti-B7-H3 antibody without containing mouse antibody components, and thus exhibits low immunogenicity in human subjects.

Also disclosed herein are the fragments of the present mAb, including scFv, Fab, Fab′, F(ab′)₂, and diabody.

According to some embodiments, the present antibody (including mAb 91H06 and its fragment) exhibits binding specificity toward B7-H3, without cross-reacting with other B7 family members. In one embodiment, the present antibody recognizes and binds to human B7-H3 (SEQ ID NO: 9). In another embodiment, the present antibody recognizes and binds to monkey B7-H3 (SEQ ID NO: 10). In another embodiment, the present antibody recognizes and binds to rat B7-H3 (SEQ ID NO: 11). In still another embodiment, the present antibody recognizes and binds to mouse B7-H3 (SEQ ID NO: 12). According to one specific embodiment, the present antibody binds to the IgV2 and/or IgC2 domain of human B7-H3, in which the IgV2 and IgC2 domain comprises the amino acid sequence of SEQ ID NO: 13.

### (ii) Immunoconjugate Comprising the Present Recombinant Antibody

According to certain embodiments of the present disclosure, the present antibody exhibits binding affinity to the B7-H3 molecule expressed on the surface of cancer cells, and efficiently triggers the internalization of cell surface B7-H3. Based on the targeting and internalizing properties, the present antibody may serve as a targeting module for delivering a therapeutic agent connected therewith to the cancer cells in a subject.

Accordingly, the second aspect of the present disclosure is directed to an immunoconjugate. Structurally, the present immunoconjugate comprises the present mAb 91H06 or a fragment thereof (e.g., an scFv), a therapeutic agent, and a linker connecting the therapeutic agent to the present mAb/antibody fragment.

Depending on desired purpose, the therapeutic agent may be a cytotoxic drug, a radioactive nuclide, a cytokine, a hormone drug, an immune stimulating agent, or an immunotherapeutic drug.

Examples of the cytotoxic drug suitable to produce the present immunoconjugate include, but are not limited to, taxanes (e.g., paclitaxel, docetaxel and cabazitaxel), alkylating agents (e.g., mechlorethamine, cyclophosphamide, melphalan, chlorambucil, ifosfamide, busulfan, N-Nitroso-N-methylurea (MNU), carmustine (BCNU), lomustine (CCNU), semustine (MeCCNU), fotemustine, streptozotocin, dacarbazine, mitozolomide, temozolomide, thiotepa, mytomycin, diaziquone (AZQ), cisplatin, carboplatin, oxaliplatin, procarbazine and hexamethylmelamine), antimetabolites (e.g., methotrexate, pemetrexed, fluorouracil, capecitabine, cytarabine, gemcitabine, decitabine, azacitidine, fludarabine, nelarabine, cladribine, clofarabine, pentostatin, thioguanine and mercaptopurine), anti-microtubule agents (e.g., tubulysin, auristatin or its derivatives, maytansinoid, vincristine, vinblastine, vinorelbine, vindesine, vinflunine, etoposide and teniposide), topoisomerase inhibitors (e.g., irinotecan, topotecan, etoposide, doxorubicin, mitoxantrone, teniposide, novobiocin, merbarone and aclarubicin), cytotoxic antibiotics (e.g., calicheamicin, pyrrolobenzodiazepine, duocarmycin, doxorubicin, daunorubicin, epirubicin, idarubicin, pirarubicin, aclarubicin, mitoxantrone, bleomycins, mitomycin C and actinomycin), plant toxins (e.g, ricin, abrin, saporin, volkensin, modeccin, gelonin and viscumin), exotoxins (e.g., Pseudomonas exotoxin and botulinum toxin, such as botulinum toxin A, botulinum toxin B, botulinum toxin C, botulinum toxin D, botulinum toxin E and botulinum toxin F), and endotoxins (e.g., diphtheria toxin). According to one exemplary example of the present disclosure, the cytotoxic drug is auristatin or a derivative thereof, e.g., MMAE or MMAF.

The radioactive nuclide, also known as radionuclide, radioisotope or radioactive isotope, may be any of yttrium-90 (⁹⁰Y), indium-111 (¹¹¹In), iodine-131 (¹³¹I), samarium-153 (¹⁵³Sm), lutetium-177 (¹⁷⁷Lu), astatine-211 (²¹¹At), bismuth-212 (²¹²Bi), actinium-225 (²²⁵Ac), radium-223 (²²³R) or thorium-227 (²²⁷Th). Preferably, the radioactive nuclide is complex with a chelator, such as ethylenediaminetetramethylenephosphonic acid (EDTMP), 1,4,7,10-1,4,7,10-tetraazacyclododecanetetramethylenephosphonic acid (DOTMP), tetraazacyclododecane-1,4,7,10-tetraacetic acid (DOTA), 1,4,7-triazacyclononane-1,4,7-triacetic acid (NOTA), 1,4,7-triazacyclononane-1,4-diacetic acid (NODA), or diethylenetriaminepentaacetic acid (DTPA).

The cytokine may by any cytokine known to stimulate immune responses, for example, interleukin (IL)-2, IL-10, IL-12, IL-15, IL-21, tumor necrosis factor (TNF)-α, interferon (IFN)-α, IFN-γ, or granulocytemacrophage colony-stimulating factor (GM-CSF).

Exemplary hormone drugs suitable to produce the present immunoconjugate include, but are not limited to, aromatase inhibitors (e.g., anastrozole, exemestane and letrozole), selective estrogen receptor modulators (SERMs; e.g., tamoxifen and raloxifene), estrogen receptor antagonists (e.g., fulvestrant and toremifene), luteinizing hormone-releasing hormone (LHRH) agonists (e.g., goserelin, leuprolide and triptorelin), anti-androgens (e.g., apalutamide, enzalutamide, darolutamide, bicalutamide, flutamide and nilutamide), CYP17 inhibitors (e.g., abiraterone and ketoconazole), progestins (e.g., medroxyprogesterone acetate and megestrol acetate), and adrenolytics (e.g., mitotane).

Examples of the immune stimulating agent (also known as immunostimulant) include, but are not limited to, acemannan, bropirimine, burdock, deoxycholic acid (DCA), echinacea, elapegademase, flavonoids (e.g., rutin, isoliquiritigenin and liquiritigenin), glatiramer acetate, oprelvekin, pegademase bovine, plerixafor, prolactin, trilaciclib, terpenes (e.g., triterpenoid), TLR7/TLR8 agonist, TLR9 agonist, Sting agonist, and NLRP agonist.

Regarding the immunotherapeutic drug, it may be an immune checkpoint inhibitor (e.g., an inhibitor of cytotoxic T-lymphocyte antigen-4 (CTLA-4), programmed cell death 1 (PD-1) or PD-L1), or an immunomodulatory agent (e.g., thalidomide and lenalidomide).

The linker connecting the present mAb/antibody fragment and therapeutic agent may be a cleavable linker or a non-cleavable linker. Exemplary cleavable linkers include, but are not limited to, protease-sensitive linkers (e.g., valine-citrulline (VC) dipeptide, valine-alanine (VA) dipeptide, valine-lysine (VL) dipeptide, valine-arginine (VR) dipeptide and glutamate-valine-citrulline (EVC) tripeptide), pH-sensitive linkers (e.g., hydrazone linker, ester linker and amide linker), and glutathione-sensitive linkers (e.g., N-Succinimidyl 4-(2-pyridyldithio) butanoate (SPDB) and N-succinimidyl-4-(2-pyridyldithio) pentanoate (SPP)). Non-limiting examples of non-cleavable linker include, maleimidocaproyl (MC), maleimidomethyl cyclohexane-1-carboxylate (MCC), and succinimidyl-4-[N-maleimidomethyl]cyclohexane-1-carboxylate (SMCC). Alternatively, the linker may be a linker known in the art to connect two functional motifs in an immunoconjugate (e.g., connecting the antibody and the payload of an ADC). A skilled artisan may select a suitable linker for producing the present immunoconjugate in accordance with intended purpose. According to one exemplary embodiment, the linker connecting the present mAb/antibody fragment and therapeutic agent comprises a polyethylene glycol (PEG) chain and a protease-sensitive linker connected to the PEG chain; preferably, the PEG chain has 1 to 10 repeats of EG units. According to one example of the present disclosure, the linker comprises a PEG chain, and a VC dipeptide linked to the PEG chain; in the embodiment, the immunoconjugate is produced in the form of “mAb/antibody fragment-PEG chain-VC dipeptide-therapeutic agent”. In another example of the present disclosure, the linker comprises a PEG chain, and a EVC tripeptide linked to the PEG chain; in the embodiment, the immunoconjugate is produced in the form of “mAb/antibody fragment-PEG chain-EVC tripeptide-therapeutic agent”.

As described above, the present mAb may be produced in the form of IgG, IgA, IgM, IgD or IgE in accordance with practical needs. According to some preferred embodiments of the present disclosure, the present mAb is in the form of IgG. In these embodiment, the therapeutic agent is linked to the Fc region of the mAb.

According to some embodiments of the present disclosure, the present immunoconjugate is produced by trimannosyl ADC technology, a platform for linking drug payload(s) to a target antibody in a site-specific manner (see, for example, WO 2018/126092 A1). In these embodiments, the recombinant antibody or antibody fragment is first modified to conjugate with one or more (e.g., 1, 2, 3, 4, 5, 6, 7, 8 or more) azide groups; the azide-modified antibody is then capable of linking to one or more (e.g., 1, 2, 3, 4, 5, 6, 7, 8 or more) dibenzocyclooctyne group (DBCO)-conjugated linker-payloads (each of which comprises a linker, a DBCO group conjugated to one end of the linker, and a therapeutic agent conjugated to the other end of the linker) via a copper-free click reaction occurred between the azide and DBCO groups. As could be appreciated, the azide or DBCO group may be alternatively substituted by different groups suitable for click chemistry, for example, an alkyne, tetrazine, or trans-cyclooctyne (TCO) group. Depending on desired purpose, the present immunoconjugate may alternatively be produced by other methods known to synthesize ADCs, for example, cysteine conjugation, lysine conjugation, disulfide re-bridging, etc. The methods for synthesizing ADCs are known in the art; hence, the detailed description is omitted herein for the sake of brevity.

According to certain exemplary embodiments of the present disclosure, the recombinant antibody/antibody fragment has four azide groups, and accordingly, four therapeutic agents (e.g., four MMAE or MMAF molecules) are conjugated to the recombinant antibody/antibody fragment. In these embodiments, the drug-to-antibody ratio (DAR) of the thus-produced immunoconjugate is about 4.

(iii) Pharmaceutical Compositions Comprising the Present Immunoconjugate

The third aspect of the present disclosure is directed to a pharmaceutical composition for the treatment of cancers. According to some embodiments, the pharmaceutical composition comprises the present immunoconjugate, and optionally, a pharmaceutically acceptable carrier.

Generally, the immunoconjugate of this invention is present at a level of about 0.1% to 99% by weight, based on the total weight of the pharmaceutical composition. In some embodiments, the immunoconjugate of this invention is present at a level of at least 1% by weight, based on the total weight of the pharmaceutical composition. In certain embodiments, the immunoconjugate is present at a level of at least 5% by weight, based on the total weight of the pharmaceutical composition. In still other embodiments, the immunoconjugate is present at a level of at least 10% by weight, based on the total weight of the pharmaceutical composition. In still yet other embodiments, the immunoconjugate is present at a level of at least 25% by weight, based on the total weight of the pharmaceutical composition.

The pharmaceutically acceptable carrier may be any pharmaceutically acceptable material or vehicle, such as a liquid or solid filler, diluent, excipient, solvent or encapsulating material, that is useful in carrying or transporting the active agents (e.g., the present mAb or immunoconjugate) from one organ, or portion of the body, to another organ, or portion of the body. The carrier must be “acceptable” in the sense of being compatible with the other ingredients of the formulation, and is selected to minimize any degradation of the active agent and to minimize any adverse side effects in the subject. Depending on desired purposes, the pharmaceutical composition of the present disclosure may further comprise one or more pharmaceutically-acceptable additives, including binder, flavoring, buffering agent, thickening agent, coloring agent, anti-oxidant, diluent, stabilizer, buffer, emulsifier, dispersing agent, suspending agent, antiseptic and the like.

The choice of a pharmaceutically acceptable carrier to be used in conjunction with the present immunoconjugate is basically determined by the way the pharmaceutical composition being administered. The pharmaceutical composition of the present invention may be administered to a subject via subcutaneous, intravenous, intraarterial, intratumoral, intraperitoneal, or intramuscular injection.

The pharmaceutical composition for administration by injection may be prepared in a sterile aqueous or non-aqueous solution, suspension, and emulsion. Examples of the non-aqueous solution include, but are not limited to, propylene glycol, polyethylene glycol, vegetable oils such as olive oil, and injectable organic esters such as ethyl oleate. Illustrative examples of the aqueous solution include water, alcoholic solution, emulsion, or suspension, such as saline and buffered media. Common parenteral vehicles include sodium chloride solution, Ringer's dextrose, dextrose, sodium chloride, lactated Ringer's, or fixed oils; whereas intravenous vehicles often include fluid and nutrient replenishes, electrolyte replenishes (such as those based on Ringer's dextrose), and the like.

### (iv) Uses of the Present Antibody, Immunoconjugate and Pharmaceutical Composition

The fourth aspect of the present disclosure pertains to a method of treating a cancer in a subject. The method comprises administering to the subject an effective amount of the immunoconjugate or pharmaceutical composition, in accordance with any aspect, embodiment of example of the present disclosure.

According to some embodiments, the present immunoconjugate is administered to the subject. In the embodiments, the subject is a mouse, in which 0.01 to 100 mg/Kg of the present immunoconjugate is administered to the subject, such as 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 mg/kg. Preferably, about 0.1 mg/kg to 50 mg/kg of the present immunoconjugate is administered to the subject. More preferably, about 1 mg/kg to 10 mg/kg of the present immunoconjugate is administered to the subject. According to one working example, about 5 mg/kg of the present immunoconjugate is administered to the subject. In one example, about 1.5 mg/kg is sufficient to elicit a therapeutic effect (i.e., inhibiting tumor growth) in the subject.

A skilled artisan may readily determine the human equivalent dose (HED) of the present immunoconjugate, based on the doses determined from animal studies provided in working examples of this application. Accordingly, the effective amount of the present mAb suitable for use in a human subject may be in the range of 1 μg/kg to 10 mg/kg body weight for human; such as 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 μg/kg, or 1, 2, 3, 4, 5, 6, 7, 8, 9 or 10 mg/kg. The dose can be administered in a single aliquot, or alternatively in more than one aliquot. The skilled artisan or clinical practitioner may adjust the dosage or regime in accordance with the physical condition of the patient or the severity of the diseases.

Depending on desired purpose, the present immunoconjugate and/or pharmaceutical composition may be administered to the subject 1 or 2 times every week, once every 2 weeks, once every 3 weeks, once every 4 weeks, once every 5 weeks, once every 6 weeks, once every 7 weeks, once every 8 weeks, or longer. The progress of this therapy is easily monitored by conventional techniques and assays. As could be appreciated, the dosing regimen can vary over time. According to certain exemplary embodiments of the present disclosure, the present immunoconjugate is administered to the subject once every week. According to another exemplary embodiment, single dose of the present immunoconjugate is sufficient to elicit a therapeutic effect (i.e., inhibiting tumor growth) in the subject.

The present immunoconjugate or pharmaceutical composition may be administered to the subject by a route selected from the group consisting of nasal, topical, transmucosal, and parenteral administration, in which the parenteral administration is any of subcutaneous, intratumoral, intramuscular, intravenous, intraarterial, or intraperitoneal injection. According to certain exemplary embodiments of the present disclosure, the present immunoconjugate or pharmaceutical composition is intravenously administered to the subject.

As would be appreciated, the present method can be applied to the subject, alone or in combination with additional therapies that have some beneficial effects on the prevention or treatment of cancers, for example, surgery, chemotherapy and/or radiation therapy. Depending on the intended/therapeutic purpose, the present method can be applied to the subject before, during, or after the administration of the additional therapies.

The cancer treatable with the present method may be any B7-H3-positive cancers (i.e., the cancer expressing B7-H3, or overexpressing B7-H3 as compared to normal cells), such as breast cancer, gastric cancer, colorectal cancer, gallbladder cancer, prostate cancer, cervical cancer, ovarian carcinoma, chronic or acute lymphocytic leukemia, bladder cancer, renal cancer, hepatocellular carcinoma, head and neck squamous cell carcinoma, glioblastoma, esophageal cancer, pancreatic cancer, oral cancer, lung cancer, melanoma or lymphoma.

Basically, the subject treatable with the present method is a mammal, for example, human, mouse, rat, guinea pig, hamster, monkey, swine, dog, cat, horse, sheep, goat, cow, and rabbit. Preferably, the subject is a human.

Based on the binding affinity and/or specificity toward the B7-H3 protein, the present mAb or antibody fragment may serve as a detection antibody for detecting cancers/cancerous cells expressing or overexpressing B7-H3. Accordingly, the fifth aspect of the present disclosure pertains to a method of making a diagnosis of a cancer in a subject by using the present mAb. In this aspect, the present mAb is preferably linked to a reporter molecule, for example, a fluorophore or enzyme. The method comprises, (a) isolating a biological sample from the subject; (b) mixing the biological sample with the present mAb; (c) detecting the expression of the reporter molecule; and (d) making the diagnosis of the cancer based on the result of step (c), in which in the case when the reporter molecule is detected in step (c), then the subject has the cancer.

Alternatively, the present mAb may serve as a capture antibody for capturing the B7-H3 expressing cancers/cancerous cells, followed by detecting with a reporter-conjugated secondary antibody (i.e., a detection antibody) that recognizes either the present m Ab (e.g., the Fc region of the mAb) or the cancerous cells.

Depending on intended purpose, the present mAb may be used in different detection assays, for example, immunohistochemistry (IHC), immunofluorescence, enzyme-linked immunosorbent assay (ELISA), flow cytometry, and western blot.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Preparation of B7-H3 Proteins

The nucleotide sequence encoding human (amino acid residues 29-466), or rat or mouse (amino acid residues 29-244) B7-H3 extracellular domain was fused with mouse Fc domain, and then constructed into pcDNA3.4 vector. The recombinant human B7-H3-mFc, rat B7-H3-mFc and mouse B7-H3-mFc proteins were respectively expressed and purified by FreeStyle™ 293 cells. The recombinant cynomolgus monkey B7-H3 protein was purchased from R&D Systems®.

Screening B7-H3 mAbs from Synthetic Human Antibody Libraries
(A) Preparation of scFv Phages for Bio-Panning

Phage-displayed scFv libraries comprising approximately 1011 antibody variants were cultured with TG1 cells in 2× yeast tryptone medium (YT medium) containing 100 μg/ml ampicillin and 2% glucose, followed by incubating with shaking at 37° C. until the OD at 600 nm reached 0.5. The helper phage was added to the culture, and then incubated in a 37° C. water bath for 30 minutes. After spinning the infected cells at 4,000 rpm for 15 minutes, the pellet was gently resuspended in 2×YTAK medium (YT medium containing 100 μg/ml ampicillin and 25 μg/ml kanamycin), and incubated with shaking at 37° C. overnight. The culture was centrifuged at 10,000 rpm for 20 minutes followed by adding ⅕ volume PEG/NaCl (20% polyethylene glycol 8000, 2.5 M NaCl) to the supernatant, and incubated at 4° C. for at least one hour. After spinning 10,000 rpm for 20 minutes, the pellet was washed by sterile water to remove bacterial debris. ⅕ volume PEG/NaCl was added to the supernatant and incubated at 4° C. for at least one hour. The pellet was washed by phosphate-buffered saline (PBS) to remove bacterial debris.

### (B) Selection of Phages

The ELISA plate was coated with human B7-H3-mFc protein, and incubated at 4° C. overnight. The plate was blocked by 300 μL of 5% skim milk (diluted by PBS) at 37° C. for 90 minutes. After washing with PBS for three times, 100 μL of 1011 to 1012 phages diluted in 5% MPBS (PBS containing 2% skim milk) were added to the plate followed by incubating at 37° C. for 90 minutes. The plate was washed by PBST (PBS containing Tween® 20) for three times, and PBS for one time. 100 μL of 100 mM trimethylamine (TEA) were added to the plate so as to elute phages, which were then neutralized by 1 M Tris (pH7.4). The thus-obtained phages were mixed with TG1 cells, and incubated at 37° C. for 30 minutes. After centrifuging at 4,000 rpm for 15 minutes, the TG1 cells were resuspended in 2×TY medium, and then plated on a 2×YTAG (yeast tryptone medium supplemented with ampicillin and glucose) plate to amplify the infected cells.

### (C) Preparation of Next Round Phages

5-6 ml of 2×YT medium containing 15% glycerol were added to the bacterial plate thereby scraping the bacteria off the plate. 50-100 μL of the scraped bacteria were cultured in 100 ml of 2×YTAG, and shaking at 37° C. until the OD at 600 nm reached 0.5. The helper phage was added to the culture, and then incubated in a 37° C. water bath for 30 minutes. After spinning the infected cells at 4,000 rpm for 15 minutes, the pellet was gently resuspended in 2×YTAK medium, and incubated with shaking at 37° C. overnight. The culture was centrifuged at 10,000 rpm for 20 minutes followed by adding ⅕ volume (8 mL) PEG/NaCl to the supernatant, and incubated at 4° C. for at least one hour. After spinning 10,000 rpm for 20 minutes, the pellet was washed by PBS to remove bacterial debris. The phage library was subjected to a multi-well microplate coated with human B7-H3-mFc protein. The phage clones displaying anti-B7-H3 scFvs were identify by ELISA screening.

### (D) Screening of B7-H3-Positive Phage by ELISA

The phage clones identified in (C) were incubated in 200 μL of 2×YTAG medium at 37° C. overnight. 50 μL of the inoculum were transferred to a 96-well plate containing 200 μL of 2×YTAG per well. The plate was incubated with shaking at 37° C. for 2 hours. 109 pfu of helper phage were added to the plate followed by incubating at 37° C. for 90 minutes. After centrifuging at 4,000 rpm for 30 minutes, the pellet was resuspended in 300 μL of 2×YTAG medium, and incubated at 30° C. overnight. The cells were centrifuged at 4,000 rpm for 30 minutes, and 100 μL of the supernatant were added to the ELISA plate, which was coated with human B7-H3-mFc protein and pre-treated with 300 μL of 2% MPBS. 90 minutes later, the plate was washed with PBST for three times. Appropriate dilution of HRP conjugated anti-M13 antibody (diluted in 2% MPBS) was added to the plated followed by developing with TMB substrate solution (3,3′,5,5′-Tetramethylbenzidine). The reaction was stopped by adding 50 μL of 1M sulphuric acid. Measuring the OD at 450 nm and 650 nm, and subtracting the value of OD₆₅₀ from the value of OD₄₅₀.

After three to four rounds of biopanning from the phage-displayed library screening, 60 scFv phage clones with significant B7-H3 affinity were selected and the coding sequences were constructed by inserting the VH and VL chains into an expression vector containing CH and CL chain, respectively. FreeStyle™ 293 cells were transfected with the constructed vector. The antibodies were purified by using Protein A Sepharose® Fast Flow. After purification, the antibodies were quantified by measuring at OD₂₈₀ nm and analyzed by reducing and non-reducing polyacrylamide gel electrophoresis (PAGE).

### ELISA Binding Assay

The binding affinity of full-length antibodies to human, monkey, mouse and rat B7-H3 were evaluated by ELISA. In brief, 96-well plate was coated with B7-H3 proteins (i.e., human B7-H3-mFc protein, cynomolgus monkey B7-H3 protein or rat B7-H3 Fc chimeric protein) in coating buffer and incubated overnight at 4° C. After washing with PBS for 3 times, the 96-well plate was inverted and tapped on clean absorbent papers to remove excess liquid. The blocking buffer (PBS containing 1% bovine serum albumin (BSA) was added to the wells, followed by incubating the plate at 37° C. for 1 hour with gentle continual shaking. After washing with PBS for 3 times, serial dilutions of anti-B7-H3 antibody (in PBS containing 1% BSA) were added into the wells. The plate was incubated at 37° C. for 1 hour with gentle continual shaking. After washing with PBS for 3 times, the secondary antibody (peroxidase-conjugated AffiniPure™ F(ab′)₂ fragment goat anti-human IgG (H+L)) was added into the wells and then incubated at 37° C. for 1 hour with gentle continual shaking. The plate was washed with PBS for 3 times, and the TMB substrate was added into each well and incubated at room temperature for 5 minutes, avoiding from light. 1 N HCl stop solution was added into each well and the absorbance was measured at 450/650 nm. The binding affinity value (EC50) of antibodies was determined by software.

### Surface Plasmon Resonance (SPR)

The binding kinetics of the present antibodies against human B7-H3 were determined by Biacore™ assay. Carboxymethylated dextran biosensor chips (CM5) were activated according to the supplier's instructions. Anti-human IgG (Fc) was diluted with immobilization buffer before injection at a flow rate of 10 μL/minute to achieve approximately 10,000 response units (RU) of coupled protein followed by the injection of 1 M ethanolamine to block unreacted groups. Human anti-B7-H3 antibodies were captured on anti-human IgG chip. For kinetics measurements, two-fold serial dilutions of human B7-H3-mFc protein (from 40 nM to 0.3125 nM) were injected in HBS-EP⁺ Biacore™ running buffer provided by the manufacturer at a flow rate of 30 μL/min. The binding affinities of the present antibodies to human B7-H3-mFc protein were determined via compensating the results with reference subtraction.

### Flow Cytometry

For the purpose of determining the binding affinity of the present antibodies to B7-H3-expressing cells, indirect immunofluorescence staining was performed. In brief, one hundred thousand of the indicated head and neck squamous cell carcinoma (HNSCC), prostate cancer, non-small cell lung cancer (NSCLC) or hepatocellular carcinoma (HCC) cells, were suspended in FACS buffer (PBS containing 0.5-1% BSA or 5-10% FBS, and 0.1% NaN3 sodium azide) and mixed with the present antibody (1 μg/well) in a 96-well U-shaped-bottom microplate at 4° C. for 60 minutes. After washing with PBS, the secondary antibody (Alexa Fluor® 647-conjugated anti-human IgG in FACS buffer) was added to the plate and incubated at 4° C. for another 60 minutes. The fluorescent intensities of cells were measured by flow cytometry and analyzed via software.

For the purpose of examining the internalization of the present antibody, indirect immunofluorescence staining was performed. In brief, cancer cells (6×10⁵), including HNSCC cells and prostate cancer cells were suspended in FACS buffer and mixed with the present antibody (6 μg/well) in a 96-well U-shaped-bottom microplate at 4° C. for 60 minutes. After washing with PBS to remove excess antibodies, 1.5×10⁵ cells were divided into 4 aliquots of tubes and incubated at 37° C. for 30, 60, 120 or 180 minutes. Next, the cells were kept on ice before staining with secondary antibody. After washing with PBS, the secondary antibody (Alexa Fluor® 647-conjugated anti-human IgG in FACS buffer) was added to the well, followed by incubating the plate at 4° C. for another 60 minutes. The fluorescent intensities of cells were measured by flow cytometry and analyzed via software.

### Cell Culture

Detroit 562 cells (a human pharyngeal squamous cell carcinoma cell line) were cultured in minimum essential medium (MEM supplemented with 0.1 mM non-essential amino acids, 1.0 mM sodium pyruvate, 0.1% lactalbumin hydrolysate and 10% fetal bovine serum (FBS).

FaDu cells (a human HNSCC cell line), DU145 cells (a human cell line derived from brain metastasis of prostate carcinoma), and HepG2 cells (a human HCC cell line) were cultured in MEM supplemented with 0.1 mM non-essential amino acids, 1.0 mM sodium pyruvate and 10% FBS. B7-H3 knock-out FaDu (FaDu/B7-H3 KO) cells were created via CRISPR/Casp9 technology.

CAL 27 cells (a human oral adenosquamous carcinoma cell line), Huh7 cells (a human HCC cell line), and A549 cells (a human NSCLC cell line) were cultured in Dulbecco's Modified Eagle Medium (DMEM) supplemented with 10% fetal bovine serum.

OECM-1 cells (a human oral cavity squamous cell carcinoma cell line), LNCaP cells (a human prostate cancer cell line derived from a metastatic lymph node lesion), H1299 cells (a human NSCLC cell line), and NCI-H520 cells (a human lung squamous cell carcinoma cell line) were cultured in RPMI supplemented with 10% FBS.

PC-3 cells (a human prostate cancer cell line) were cultured in Ham's F12K medium containing 2 mM L-glutamine, 1.5 g/L sodium bicarbonate and 7% FBS.

HA59T cells (a human HCC cell line) were cultured in DMEM containing 4 mM L-glutamine, 4.5 g/L glucose, 0.1 mM non-essential amino acids and 10% FBS.

HuT78 cells (a human cutaneous T-lymphocyte cell line) were cultured in Iscove's Modified Dulbecco's Medium (IMDM) supplemented with 20% FBS.

Ba/F3 cells (a murine interleukin-3 dependent pro-B cell line) were cultured in RPMI supplemented with 10% FBS and 10 ng/ml mouse IL-3.

EMT6 cells (a murine mammary carcinoma cell line) were cultured in Waymouth's medium (MB 752/1) containing 2 mM L-glutamine and 15% FBS.

ID8 cells (a murine ovarian surface epithelial cell line) were cultured in DMEM supplemented with 4% FBS and 1×ITS (a mixture of recombinant human insulin, human transferrin, and sodium selenite).

### Antibody-Drug Conjugate (ADC)

The present ADCs were produced via tri-mannosyl antibody drug conjugate platform. Specifically, 0.04 mL linker-payloads (10 mM in dimethyl sulfide (DMSO) or dimethylacetamide (DMA)), along with 0.12 mL DMSO or DMA, was slowly added into the solution of mAb-4 Azide (Az) (0.4 mL, 5 mg/mL) in 2-(N-morpholino) ethanesulfonic acid (MES) buffer (pH6.5). The reaction mixture was stirred under argon at 37° C. for 18 hours. The thus-formed antibody-drug conjugates were desalted and concentrated by using the centrifugal filter with 30 kDa nominal molecular weight limit (NMWL) in MES buffer (pH6.5) so as to produce 91H06-MMAE and 91H06-MMAF, respectively. In the present study, the linker for connecting the mAb 91H06 and drug payload (MMAE or MMAF) comprised a PEG linker, and a protease-sensitive linker of valin-citrulline or glutamate-valine-citrulline linked to the PEG chain. The drug-to-antibody ratio (DAR) of ADCs was measured by liquid chromatography mass spectrometry (LC-MS). The DAR of 91H06-MMAE and 91H06-MMAF were between 3.5 and 3.9.

### Cytotoxicity Assay

The cells were seeded in triplicated into 96-well plate overnight. Serial dilutions of the present ADC (final concentrations: 500 nM to 0.025 nM) were prepared and added into the wells. After incubating at 37° C. for 120 hours, CellTiter-Glo® reagent were prepared according to the manufacture's manual and added into each well, avoiding the light. The plate was shacked for 2 minutes on an orbital shaker to mix the solution and induce cell lysis. After incubating at room temperature for 10 minutes, the luminescence values were record and calculated by software.

### Animal Study

The in vivo anti-tumor efficacy of 91H06-MMAE was evaluated on Detroit 562 xenograft model and FaDu xenograft model in NOD SCID mice. Male NOD SCID mice at age of 6-8 weeks were used in the study. Five mice were housed in each cage. All animals were hosted in the animal facility with a 12-h light/12-h dark cycle at 19-25° C. Animals had free access to rodent pellet foods and water ad libitum.

Detroit 562 tumor cells (3×10⁶ cells) or FaDu tumor cells (1×10⁶ cells) mixed with Matrigel® (1:1, v/v) in a dose volume of 0.1 mL were subcutaneously (SC) injected to the right front flank of mice for tumor growth. When the mean tumor volume reached approximately 160 mm³ (in the Detroit 562 tumor model) or 100 mm³ (in the FaDu tumor model), tumor-bearing mice were randomly divided to 3 groups, in which each group consisted of 5 mice. The 91H06-MMAE or vehicle solution (25 mM MES buffer, pH6.5) was intravenously administrated to the tumor-bearing mice. The initial dosing day was denoted as Day 0. Animals received the vehicle solution served as the disease (vehicle) control group for calculation of tumor growth inhibition (TGI) rate. The animals of experimental groups was administered with 91H06-MMAE at 1.5 mg/kg (mpk) or 5 mg/kg (mpk) once per week for three consecutive weeks in Detroit 562 tumor model; or administered with single dose of 91H06-MMAE at 1.5 mg/kg (mpk) or 5 mg/kg (mpk) in FaDu tumor model.

In the study of evaluating the anti-tumor effect of 91H06-MMAE, Detroit 562 tumor cells (3×10⁶ cells) mixed with Matrigel® (1:1, v/v) in a dose volume of 0.1 mL were subcutaneously injected to the right front flank of mice for tumor growth. When the mean tumor volume reached approximately 1,500 mm³, the tumor-bearing mice were intravenously administrated with 91H06-MMAE at 5 mg/kg (mpk) once per week for three consecutive weeks.

The tumor volumes, body weights, mortality, and signs of overt toxicity were monitored and recorded three times weekly. Tumor volumes (mm³) were measured using digimatic calipers and calculated according the formula: TV=(W2 xL)/2; where W=width and L=length in diameter (mm) of the tumor. The percentages of TGI were calculated using the following formula: TGI=[1−(Tx−T0/Cx−C0)]×100%, where Tx and Cx represented the mean tumor volumes on Day X of the treatment group and the control group, respectively.

### Example 1 Characterization of mAb 91H06

### 1.1 Protein Binding Affinity

The binding activity of mAb 91H06 or Ifinatamab (an B7-H3 directed antibody, serving as a positive control) to human, monkey, mouse or rat B7-H3 was evaluated in this example. As described in “Materials and Methods” of the present disclosure, the mAb 91H06 was added to the plate coated with human B7-H3-mFc, cynomolgus monkey B7-H3, mouse B7-H3 mFc or rat B7-H3 mFc chimeric protein, and the binding activity therebetween was determined by ELISA.

The results indicated that the present mAb 91H06 exhibited binding affinity towards human, monkey, rat and mouse B7-H3 with EC50<10-10 (Table 1). In contrast, Ifinatamab only recognized human and monkey B7-H3 (Table 1).

TABLE 1
Binding affinity of Ifinatamab or mAb
91H06 to specified B7-H3 protein

| | Human | | | |
|---|---|---|---|---|
| | B7-H3 | Monkey | Rat B7-H3 | |
| | EC₅₀, | B7-H3 | EC₅₀, | Mouse B7-H3 |
| Antibody | Molar | EC₅₀, Molar | Molar | EC₅₀, Molar |
| Ifinatamab | 3.556E−011 | 1.272E−010 | — | — |
| mAb 91H06 | 1.864E−011 | 2.443E−011 | 6.008E−011 | 7.673e−011 |

The binding kinetic of the mAb 91H06 was further examined by Biacore™ assay. The data of Biacore™ assay confirmed the cross-species reactivity of mAb 91H06 against human, monkey, rat and mouse B7-H3 (Table 2).

TABLE 2
Binding affinity of mAb 91H06 to specified B7-H3 protein

| species | | | | | |
|---|---|---|---|---|---|
| cross- | mAb 91H06 | | | | |
| reactivity | Kₐ | K_{d} | K_{D} | Rmax(RU) | Chi²(RU²) |
| Human B7-H3 | 6.856E+5 | 1.558E−4 | 2.272E−10 | 155.3 | 6.28 |
| Monkey B7-H3 | 6.139E+5 | 2.892E−4 | 4.712E−10 | 99.18 | 3.67 |
| Mouse B7-H3 | 2.471E+5 | 5.015E−4 | 2.029E−9 | 93.14 | 0.627 |
| Rat B7-H3 | 1.784E+4 | 2.210E−4 | 1.239E−9 | 116.9 | 1.32 |

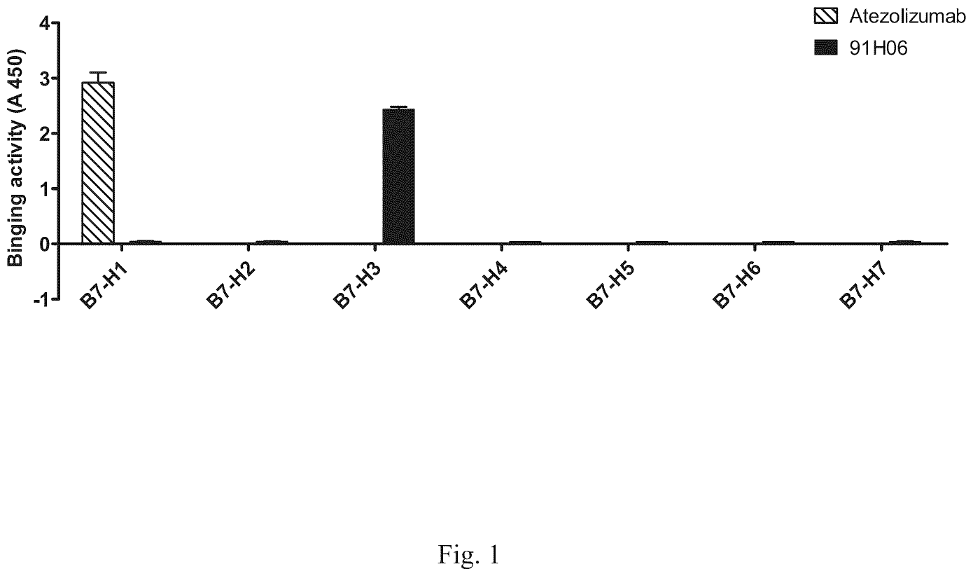

It has been reported that human B7-H3 shares sequence homology with B7-H4 (31%) and other members of the B7 family (24 to 31%). Accordingly, whether the present mAb would cross-react with other B7 proteins was also evaluated in this example. The data indicated that the present mAb 91H06 selectively bound to B7-H3, but not other members of the B7 family (FIG. 1). 1.2 Cell binding affinity

The binding activities of the present mAb to various human cancer cells were examined in the example. The data indicated that the mAb 91H06 was capable of recognizing and binding to different HNSCC cell lines, including Detroit 562, CAL 27, FaDu and OECM-1 cells; and different prostate cancer cell lines, including DU 145, LNCaP and PC-3 cells (Table 3). In addition to HNSCC and HCC cell lines, the mAb 91H06 also exhibited binding affinity to different HCC cell lines, including HepG2, HA59T and Huh7 cells; and different NSCLC cell lines, including A549, H1299 and NCI-H520 cells (Table 3). It was noted that the binding activities of the mAb 91H06 to tested cancer cells were comparable to that of the control antibody Ifinatamab, suggesting that the mAb 91H06 was effective in targeting B7-H3-expressing cancer cells.

TABLE 3
The cell binding activities of mAb 91H06
to the indicated cancer cells.

MFI value

| B7-H3 status | Cell line | IgG | 91H06 |
|---|---|---|---|
| Positive | FaDu | <600 | 11535 |
| | Detroit 562 | <600 | 14878 |
| | CAL 27 | <600 | 10962 |
| | OECM-1 | <600 | 8419 |
| | DU145 | <600 | 23205 |
| | LNCaP | <600 | 18512 |
| | PC-3 | <600 | 7942 |
| | HepG2 | <600 | 9883 |
| | HA59T | <600 | 19501 |
| | Huh7 | <600 | 27647 |
| | A549 | <600 | 10503 |
| | H1299 | <600 | 6016 |
| | NCI-H520 | <600 | 13725 |
| Low/Knock-out | HuT78 | <600 | <600 |
| | FaDu/B7-H3 KO | <600 | <600 |

Further, cancer cell line (i.e., HuT78 cells) with low B7-H3 expression and B7-H3 knock out cancer cell line (i.e., B7-H3 knock-out FaDu cells) were also examined in this example. The data indicated that the mAb 91H06 did not bind to B7-H3 negative cancer cells (Table 3), demonstrating the binding specificity of the mAb 91H06 towards cancer cells with high B7-H3 expression level.

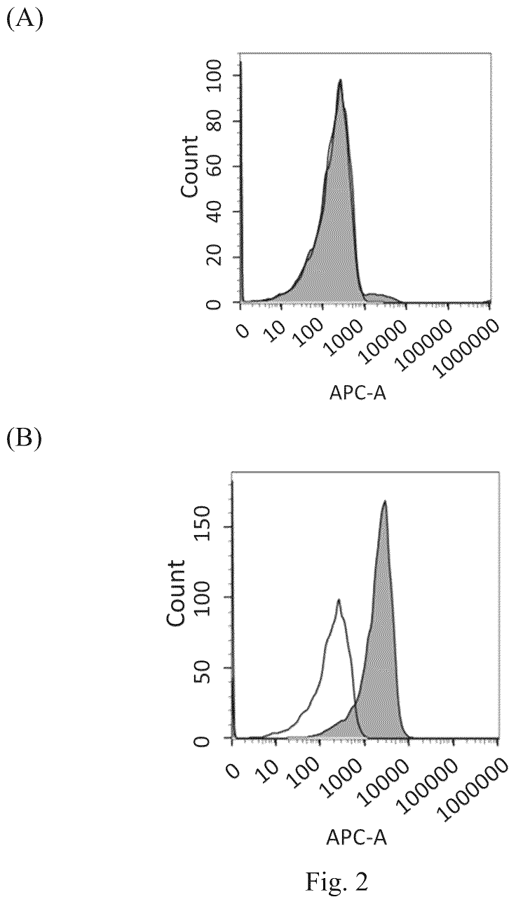

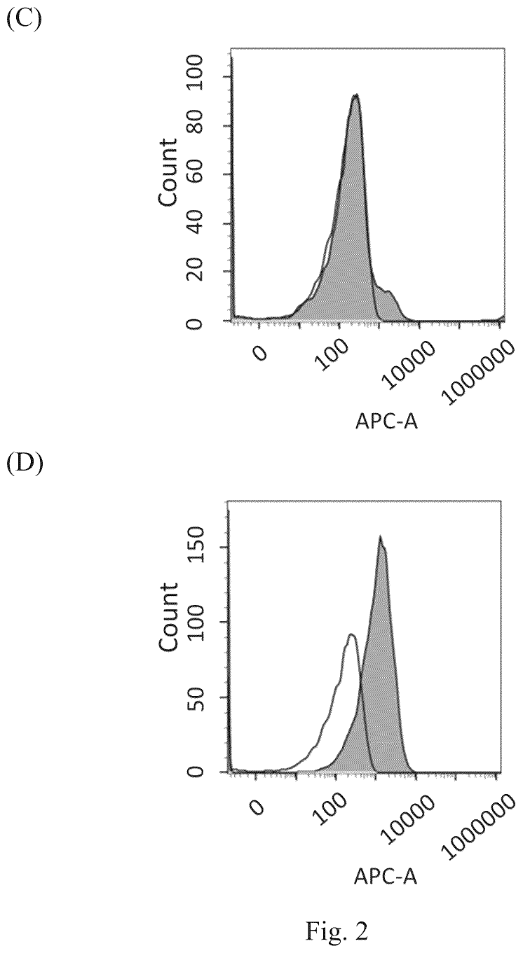

To confirm the cross-species reactivity of the present mAb, the binding activities of the mAb 91H06 to rodent cells were further examined in the example. According to the data of flow cytometry analysis, compared to the benchmark Ifinatamab which did not exhibit binding affinity to rodent cells (Panels (A) and (C) of FIG. 2), the mAb 91H06 was capable of recognizing and binding to rodent cell lines with B7-H3 expression including EMT6 and ID8 cells (Panels (B) and (D) of FIG. 2), but not to B7-H3 negative Ba/F3 cells (data not shown). The data demonstrated that the present mAb 91H06 exhibited cross-species reactivity to B7-H3 positive rodent cells.

### 1.3 Competitive Binding Assay

It is well accepted that antibodies binding to different epitopes of same target might result in different biological activities (e.g., binding mediated internalization). Therefore, competitive binding assay with the present mAb 91H06 and Ifinatamab was carried out to determine the relative position of epitopes of B7-H3 protein. The data of Table 4 indicated that the presence of Ifinatamab (4 μg/mL) altered the binding affinity of Ifinatamab-biotin towards B7-H3 protein; by contrast, the present of mAb 91H06 did not affect the interaction of Ifinatamab-biotin and B7-H3 protein. The data suggested that the mAb 91H06 did not compete with Ifinatamab for B7-H3 binding, and the B7-H3 epitope recognized by the mAb 91H06 was different from that recognized by Ifinatamab.

TABLE 4
Binding affinity of Ifinatamab-biotin to B7-H3 protein
in the absence or presence of Ifinatamab or mAb 91H06

| | Ifinatamab-biotin* |
|---|---|
| Compete Ab | EC₅₀, Molar |
| — | 3.312E−011 |
| Ifinatamab | 1.785E−009 |
| mAb 91H06 | 3.461E−011 |

*Ifinatamab-biotin: Ifinatamab conjugated with a biotin molecule.

### 1.4 Cellular Internalization

ADCs are a promising cancer treatment, in which a payload (e.g., cytotoxic drug) is coupled to an antibody (e.g., anti-H7-H3 mAb), which directs and delivers the payload to the cancer cells expressing corresponding antigen (e.g., B7-H3) so as to minimize off-target toxicities and/or side-effects in a subject. A key requirement for the ADCs is the ability of the antibody to bind to the antigen on cancer cells and being internalized. The internalized ADC is then processed in the lysosome or the late endosome to release the active cytotoxin in the cancer cells. Since cellular internalization is a prerequisite for ADC function, the antibody-triggered internalization should be extensively evaluated to ensure its proper subcellular function. Overall, for an ideal ADC design, selecting an antibody to target a novel antigen with high affinity and specificity is highly desirable. In addition, the target antigen must predominantly be expressed on the surface of the target cells (e.g., cancer cells) with minimal presence on the healthy cells. Further, a desired ADC should undergo rapid internalization to drive the delivery and release of cytotoxic payload inside the cancer cells. Accordingly, whether the present mAb would induce cellular internalization was determined in the example.

The treatment of the present mAb 91H06 triggered surface B7-H3 internalization in Detroit 562 cells, in which the cellular internalization was significantly faster than the control antibody Ifinatamab (Table 5). Similar results were observed in CAL 27 and FaDu cells (Tables 6 and 7).

TABLE 5
Internalization of mAb 91H06 in Detroit 562 cells

| | Normalized mean fluorescence intensity in remaining (%) | |
|---|---|---|
| Time (hr) | Ifinatamab | 91H06 |
| 0.0 | 100 | 100 |
| 0.5 | 93 | 57 |
| 1.0 | 88 | 42 |
| 3.0 | 71 | 26 |

TABLE 6
Internalization of mAb 91H06 in Cal27 cells

| | Normalized mean fluorescence intensity in remaining (%) | |
|---|---|---|
| Time (hr) | Ifinatamab | 91H06 |
| 0.0 | 100 | 100 |
| 0.5 | 93 | 58 |
| 1.0 | 80 | 41 |
| 3.0 | 72 | 25 |

TABLE 7
Internalization of mAb 91H06 in FaDu cells

| | Normalized mean fluorescence intensity in remaining (%) | |
|---|---|---|
| Time (hr) | Ifinatamab | 91H06 |
| 0.0 | 100 | 100 |
| 0.5 | 98 | 82 |
| 1.0 | 97 | 69 |
| 3.0 | 97 | 55 |

Further, the treatment of the present mAb 91H06 also induced surface B7-H3 internalization in DU 145 and LNCaP cells (Tables 8 and 9), in which the level of internalization was also superior to the control antibody Ifinatamab.

TABLE 8
Internalization of mAb 91H06 in DU145 cells

| | Normalized mean fluorescence intensity in remaining (%) | |
|---|---|---|
| Time (hr) | Ifinatamab | 91H06 |
| 0.0 | 100 | 100 |
| 0.5 | 85 | 63 |
| 1.0 | 78 | 49 |
| 3.0 | 68 | 31 |

TABLE 9
Internalization of mAb 91H06 in LNCaP cells

| | Normalized mean fluorescence intensity in remaining (%) | |
|---|---|---|
| Time (hr) | Ifinatamab | 91H06 |
| 0.0 | 100 | 100 |
| 0.5 | 89 | 76 |
| 1.0 | 87 | 68 |
| 3.0 | 60 | 48 |

These data suggested that the present mAb 91H06 was effective in inducing antigen-triggered internalization in cancer cells, and thus may serve as a targeting module to construct an ADC for cancer treatment.

### Example 2 Characterization of the Present ADCs

The mAb was conjugated with MMAE in accordance with the procedures described in “Materials and Methods” of the present disclosure. According to the analytic results, the thus-produced 91H06-MMAE exhibited high purity (>95%) and consistent drug-to-antibody ratio (DAR˜4) (data not shown).

The data of SPR indicated that both the parental mAb 91H06 and the 91H06-MMAE exhibited high binding affinity to B7-H3 protein, in which the association rate constant (kₐ), dissociation rate constant (kₐ) and binding kinetics dissociation constant (KD) of mAb 91H06 were respectively 6.86E+5, 1.56E−4 and 2.27E−10, and the kₐ, kₐ and KD of 91H06-MMAE were respectively 6.43E+5, 1.55E−4 and 2.4E−10. The data of flow cytometry analysis further confirmed that the present 91H06-MMAE maintained high cell binding activity and specificity to CAL 27 and Detroit 562 cells (data not shown).

The cytotoxicity capability of the present ADC (i.e., 91H06-MMAE) was further investigated. As the results summarized in Table 10, the 91H06-MMAE exhibited cytotoxic activity to the cancer cells with high B7-H3 expression level, including oral squamous cell carcinoma CAL 27 cells, pharyngeal carcinoma Detroit 562 cells, hypopharyngeal carcinoma FaDu cells, and prostate cancer LNCaP cells; while no obvious cytotoxicity was detected in the cancer cells with low B7-H3 expression level (i.e., HuT78 cells) and B7-H3 knock-out cancer cells (i.e., FaDu/B7-H3 KO cells). The cytotoxicity results were consistent with the expression level of cell surface B7-H3 in the cancer cell lines as measured in Example 1.2.

TABLE 10
Cytotoxic activity (IC₅₀) of 91H06-MMAE
to B7-H3 positive cancer cell lines

| | Cytotoxicity activity (IC₅₀, nM) | | | | | |
|---|---|---|---|---|---|---|
| | | | | | | FaDu/ |
| ADC | CAL 27 | Detroit 562 | FaDu | LNCaP | HuT78 | B7-H3 KO |
| 91H06- | <50 | <50 | <50 | <50 | >500 | >500 |
| MMAE | | | | | | |

In addition to MMAE, the present mAb were also conjugated with different cytotoxic payloads (i.e., MMAF) which possess higher cytotoxicity than MMAE. As the results summarized in Table 11, the treatment of tested ADCs (i.e., 91H06-MMAF) also significantly killed oral squamous cell carcinoma CAL 27 cells, pharyngeal carcinoma Detroit 562 cells, hypopharyngeal carcinoma FaDu cells, and prostate cancer LNCaP cells, but not to HuT78 and FaDu/B7-H3 KO cells. Moreover, the cytotoxicity of 91H06-MMAF was higher than that of 91H06-MMAE. These findings were consistent with not only cell surface B7-H3 expression level but also the potency of different toxic payloads. Taken together, the highly potent and selective cytotoxicity of 91H06-based ADC suggested that the present 91H06 was an ideal monoclonal antibody in ADC drug development for treating B7-H3 positive cancers.

TABLE 11
Cytotoxic activity (IC₅₀) of 91H06-MMAF
to B7-H3 positive cancer cell lines

| | Cytotoxicity activity (IC₅₀, nM) | | | | | |
|---|---|---|---|---|---|---|
| | | | | | | FaDu/ |
| ADC | CAL 27 | Detroit 562 | FaDu | LNCaP | HuT78 | B7-H3 KO |
| 91H06- | <10 | <10 | <10 | <10 | >500 | >500 |
| MMAF | | | | | | |

These results demonstrated that the mAb 91H06 could be conjugated to different toxic payloads and efficiently killing various types of cancer cells.

### Example 3 In Vivo Efficacy of 91H06-MMAE in Mouse Xenograft Model

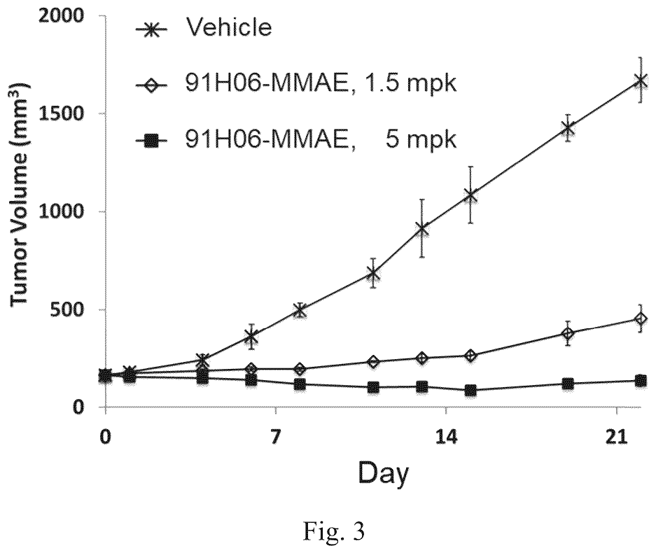

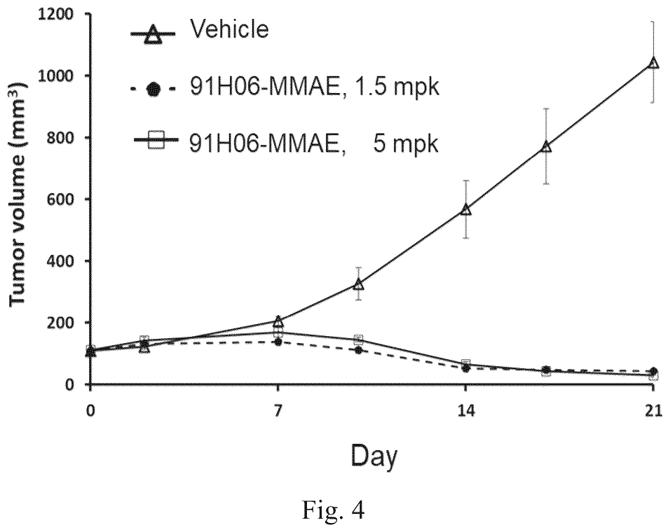

The anti-tumor effect of the present ADC in animal model was examined in this example. The data of FIGS. 3 and 4 indicated that compared to the control group, the administration of 1.5 mpk and 5 mpk 91H06-MMAE significantly suppressed tumor growth in the Detroit 562 xenograft model (FIG. 3) and FaDu xenograft model (FIG. 4). The tumor growth inhibition (TGI) rates of the present 91H06-MMAE at the dosages of 1.5 mpk and 5 mpk were respectively higher than 80% and 100% in Detroit 562 xenograft model (Table 12), and were higher than 100% in FaDu xenograft model (Table 13). No body weight loss or abnormality was detected in 91H06-MMAE treated animals (data not shown).

TABLE 12
Anti-tumor activity of the indicated treatments
in Detroit 562 xenograft model

| Treatment | TGI % |
|---|---|
| Vehicle, 10 mL/kg | — |
| 91H06-MMAE, 1.5 mpk* | >80 |
| 91H06-MMAE, 5 mpk** | >100 |

*1.5 mpk: The mice administered with 1.5 mg/kg of 91H06-MMAE once per week for three consecutive weeks.
**5 mpk: The mice administered with 5 mg/kg of 91H06-MMAE once per week for three consecutive weeks.

TABLE 13
Anti-tumor activity of the indicated
treatments in FaDu xenograft model

| Treatment | TGI % |
|---|---|
| Vehicle, 10 mL/kg | — |
| 91H06-MMAE, 1.5 mpk* | >100 |
| 91H06-MMAE, 5 mpk** | >100 |

*1.5 mpk: The mice administered with single dose of 91H06-MMAE at 1.5 mg/kg.
**5 mpk: The mice administered with single dose of 91H06-MMAE at 5 mg/kg.

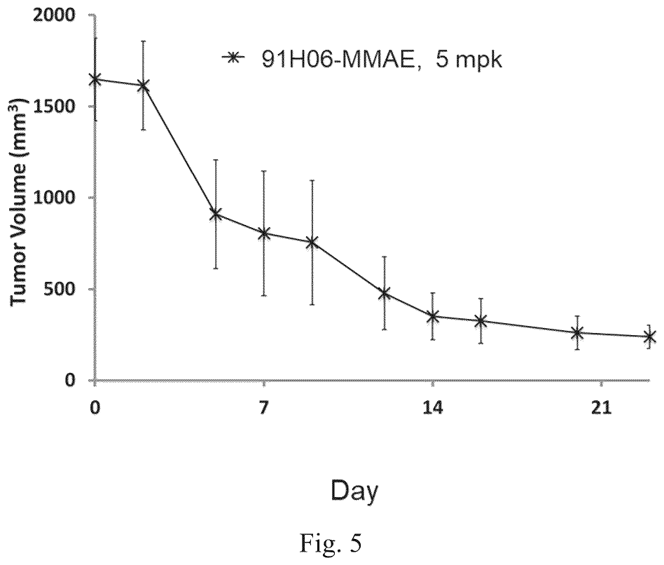

Notably, the administration of the 91H06-MMAE at 5 mg/kg induced significant tumor regression in mice bearing large tumor with mean tumor size more than 1500 mm³ (FIG. 5). No body weight loss or abnormality was detected during study period (data not shown).

In conclusion, the present disclosure provides a novel mAb 91H06. According to the examples of the present disclosure, the mAb 91H06 exhibited a high binding affinity and specificity to B7-H3, and was useful in producing an immunoconjugate (i.e., ADC) for treating B7-H3 positive cancers, even in large established tumors.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A B7-H3 targeting recombinant antibody or a fragment thereof, comprising a heavy chain variable (VH) domain and a light chain variable (VL) domain, wherein the VH domain comprises a first heavy chain complementarity determining region (CDR-H1), a second heavy chain CDR (CDR-H2) and a third heavy chain CDR (CDR-H3), and the VL domain comprises a first light chain CDR (CDR-L1), a second light chain CDR (CDR-L2) and a third light chain CDR (CDR-L3), wherein
the CDR-H1, CDR-H2 and CDR-H3 respectively comprise the amino acid sequences of SEQ ID NOs: 1, 2 and 3, and the CDR-L1, CDR-L2 and CDR-L3 respectively comprises the amino acid sequences of SEQ ID NOs: 4, 5 and 6.

2. The B7-H3 targeting recombinant antibody or the fragment thereof according to claim 1, wherein
the VH domain and VL domain respectively comprise the amino acid sequences at least 85% identical to SEQ ID NOs: 7 and 8.

3. The B7-H3 targeting recombinant antibody or the fragment thereof according to claim 2, wherein
the VH domain and VL domain respectively comprise the amino acid sequences 100% identical to SEQ ID NOs: 7 and 8.

4. An immunoconjugate comprising the B7-H3 targeting recombinant antibody or the fragment thereof according to claim 1, a therapeutic agent, and a linker connecting the therapeutic agent to the B7-H3 targeting recombinant antibody or the fragment thereof.

5. The immunoconjugate of claim 4, wherein the therapeutic agent is a cytotoxic drug, a radioactive nuclide, a cytokine, a hormone drug, an immune stimulating agent, an immunotherapeutic drug, or a combination thereof.

6. The immunoconjugate of claim 5, wherein the cytotoxic drug is selected from tubulysin, auristatin, maytansinoid, calicheamicin, pyrrolobenzodiazepine, duocarmycin, Pseudomonas exotoxin, diphtheria toxin, botulinum toxin A, selected from the group consisting of botulinum toxin B, botulinum toxin C, botulinum toxin D, botulinum toxin E, botulinum toxin F, lysine, abrin, saporin or a derivative thereof.

7. The immunoconjugate of claim 6, wherein the cytotoxic drug is auristatin or a derivative thereof.

8. The immunoconjugate of claim 7, wherein the cytotoxic drug is monomethyl auristatin E (MMAE).

9. A method of treating a cancer in a subject, comprising administering to the subject an effective amount of the immunoconjugate of claim 4.

10. The method of claim 9, wherein the cancer is breast cancer, gastric cancer, colorectal cancer, gallbladder cancer, prostate cancer, cervical cancer, ovarian carcinoma, chronic or acute lymphocytic leukemia, bladder cancer, renal cancer, hepatocellular carcinoma, head and neck squamous cell carcinoma, glioblastoma, esophageal cancer, pancreatic cancer, oral cancer, lung cancer, melanoma or lymphoma.

11. The method of claim 10, wherein the subject is a human.
